(19) United States
(12) Plant Patent Application Publication
(10) Pub. No.: US 2012/0233732 P1
(43) Pub. Date: Sep. 13, 2012

(54) Aster plant named 'Mystery Lady Marlene'

(50) ;

(75) Inventor: Bent Juhl Jensen, Malling (DK)

(73) Assignee: GARTNERIET RAAHOJ

(21) Appl. No.: 13/064,118

(22) Filed: Mar. 7, 2011

(51) Int. Cl. A01H 5/00

(52) U.S. Cl. PLT/355

(57) ABSTRACT

A new and distinct cultivar of Aster plant named ‘MYSTERY LADY MARLENE’, characterized by its upright and somewhat outwardly spreading plant habit, dark green foliage, freely flowering habit, decorative-type inflorescences with purple-violet ray florets, and almost no disk florets (i.e., close to 100% “filled” capitulae.)

### LATIN NAME OF GENUS AND SPECIES OF THE PLANT CLAIMED

Symphyotricium novi-belgii

### VARIETY DENOMINATION

‘Mystery Lady Marlene’

### BACKGROUND OF THE INVENTION

The present invention relates to a new and distinct cultivar of Aster plant, botanically known as Symphyotricium novi-belgii, and hereinafter referred to by the name ‘Mystery Lady Marlene’.

The new Aster is a product of a planned breeding program conducted by the Inventor in Malling, Denmark. The objective of the breeding program is to create new potted Aster cultivars with uniform plant growth habit, desirable floret colors, and good postproduction longevity.

The new Aster originated from a cross-pollination made by the Inventor in Malling, Denmark in September 2007, between the Aster cultivar Victoria Mathilde (U.S. Plant Pat. No. 19,930) as seed parent, and RH05-108-05 (unpatented) as pollen parent. The new Aster was discovered and selected by the Inventor as a single flowering plant within the progeny of the stated cross-pollination grown in a controlled environment in Malling, Denmark. The selection of this plant was based on its uniform plant growth habit and desirable inflorescence form and ray floret color.

Asexual reproduction of the new Aster by vegetative tip cuttings was first conducted in Malling, Denmark during June, 2008. Asexual reproduction by cuttings has shown that the unique features of this new Aster are stable and reproduced true to type in successive generations.

### SUMMARY OF THE INVENTION

The cultivar ‘MYSTERY LADY MARLENE’ has not been observed under all possible environmental conditions. The phenotype may vary somewhat with variations in environment such as temperature, day-length, and/or light level, without, however, any variance in genotype.

The following traits have been repeatedly observed and are determined to be the unique characteristics of ‘MYSTERY LADY MARLENE’. These characteristics in combination distinguish ‘MYSTERY LADY MARLENE’ as a new and distinct Aster cultivar:

1. Upright and somewhat outwardly spreading plant habit;

2. Dark green foliage;

3. Freely flowering habit;

4. Decorative-type inflorescences with purple-violet colored ray florets; and

5. Almost no disk florets (i.e., close to 100% “filled” capitulae).

Plants of the instant cultivar ‘MYSTERY LADY MARLENE’ differ primarily from plants of the seed parent, the patented (U.S. Plant Pat. No. 19,930) cultivar ‘Victoria Mathilde’, in the following characteristics:

1. plants of ‘MYSTERY LADY MARLENE’ are more compact than plants of ‘Victoria Mathilde’;

2. plants of the ‘MYSTERY LADY MARLENE’ have more ray florets per inflorescence (about 270) than plants of the cultivar ‘Victoria Mathilde’ (about 150); and

3. plants of the ‘MYSTERY LADY MARLENE’ have purple-violet (RHS N82A) ray florets, whereas plants of ‘Victoria Mathilde’ have violet-blue (RHS 90A) ray florets.

### BRIEF DESCRIPTION OF THE PHOTOGRAPHS

The accompanying colored photographs illustrate the overall appearance of the new Aster ‘MYSTERY LADY MARLENE’, showing the colors as true as it is reasonably possible to obtain in colored reproductions of this type. Colors in the photographs may differ slightly from the color values cited in the detailed botanical description, which more accurately describe the actual colors of ‘MYSTERY LADY MARLENE’.

FIG. 1 shows a side perspective view of a typical flowering plant of ‘MYSTERY LADY MARLENE’.

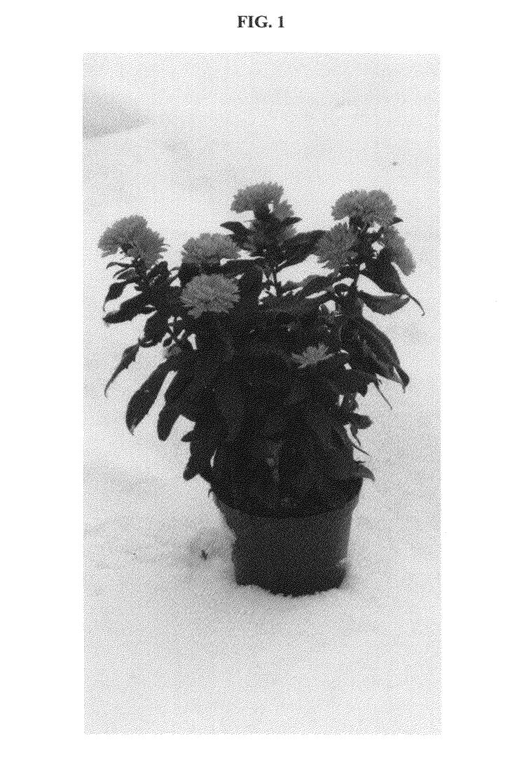

FIG. 2 shows a close up view of the upper surfaces of typical inflorescences and leaves of ‘MYSTERY LADY MARLENE’.

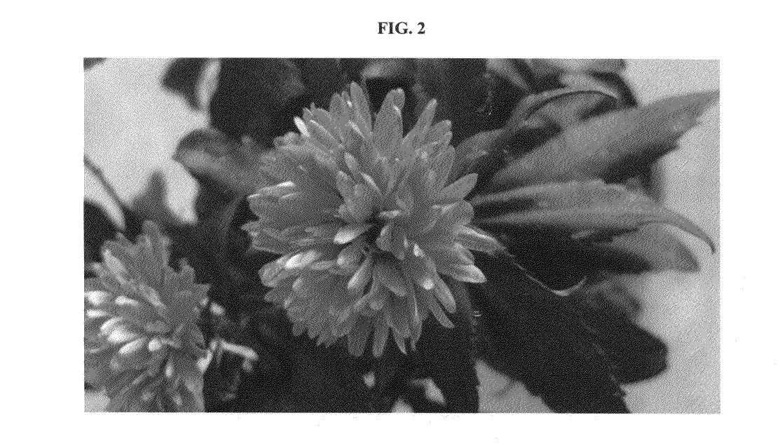

### DETAILED BOTANICAL DESCRIPTION

In the following description, color references are made to The Royal Horticultural Society Colour Chart, 4^{th} Edition, except where general terms of ordinary dictionary significance are used. The aforementioned photographs, following observations and measurements describe plants grown and flowered during the autumn in Malling, Denmark, in a glass-covered greenhouse and under conditions which approximate those generally used in commercial potted Aster production. During the production of these plants, day and night temperatures were about 18 to 20 degree C.

3 cuttings were planted in 9.5 cm pots and pinched twice and treated three times with growth retardant daminozide. Plants were about 10 weeks old when the photographs and the botanical description were taken.
Classification:
   Botanical.—Symphyotricium novi-belgii.
Parentage:
   Female (seed) parent.—Aster cultivar named ‘Victoria Mathilde’ (Patented, U.S. Plant Pat. No. 19,930).
   Male (pollen) parent.—Aster cultivar referred to as ‘RH05-108-05’ (Breeder Reference, unpatented).
Propagation:
   Type cutting.—Terminal vegetative cuttings.
   Time to initiate roots.—Summer: About 12 days at 21° C. Winter: About 13 days at 21° C.
   Root habit.—Freely branching.
   Root color.—White, close to RHS 155D.
Plant description:
   Form.—Herbaceous, decorative-type potted plant, with stems upright and somewhat outwardly spreading, inverted conical plant habit, appropriate for 9-15 cm containers. Campanulate flowers in racemes. Freely branching with lateral branches forming at every node; dense and bushy. Freely branching, somewhat brittle stems.
   Crop time.—After rooting, about 14 weeks are required to produce finished flowering plants in 11 cm pots.
   Plant height (from pot rim to top of plant plane).—About 13-14 cm.
   Plant spread (width).—About 20 cm.
   Lateral branches.—Length: About 5-8 cm. Diameter: About 2 mm. Strength: Strong. Angle: Mostly erect. Texture: Glabrous. Color: RHS 147B, yellow-green.
Foliage description:
   Leaves.—Arrangement: Alternate, sessile, decurrent. Overall shape: Older Leaves: Spatulate Younger Leaves: Oblanceolate Apex shape: Acute. Base shape: Decurrent, auriculate, ½ clasping. Length: Up to 5 cm. Width: Up to 15 mm. Margin: Serrate. Texture (both sides): Glabrous, smooth, leathery. Pubescence: Few, short, stiff hairs on abaxial side and along veins and indentations of margin. Foliage Color: Mature: Upper surface: Green, RHS 139A. Under Surface: Gray-green, RHS 191A-B. Immature: Upper surface: Green, RHS 138A. Under Surface: Gray-green, RHS 191A-B. Venation: Color: Upper surface: Green, RHS 139A-C. Under surface: Gray-green, RHS 191A.
Inflorescence description:
   Inflorescence arrangement and shape.—Upright decorative-type capitulate inflorescences with obelliptic ray florets and few tubular disk florets, terminal (or few axillary) in tight corymbose stands, disk and ray florets develop acropetally on a capitulum. Persistent.
   Natural flowering season.—Late summer. 5 weeks of long day treatments (20 hours) induces budding.
   Flower longevity.—Inflorescences maintain good color and substance for about five weeks in an interior environment, longer if temperatures are kept below 20 degree C.
   Fragrance.—None.
   Inflorescence size.—Height: About 1.8 cm. Diameter: About 4.5 cm.
   Number of inflorescences per plant.—About 30.
   Buds.—Length: About 5 mm. Diameter: About 7 mm. Shape: Globular. Color: Yellow-green, RHS147C.
   Ray florets.—Arrangement: In 9 to 12 whorls. Quantity per inflorescence: About 250-300. Length: About 13 mm. Width: About 4 mm. Overall Shape: Oblong, slightly incurved. Apex: Rounded to slightly retuse. Base: Attenuate. Margin: Entire. Texture: Smooth, glabrous. Orientation: Initially upright, later more horizontal. Aspect: Straight to slightly involute Color when opening: Upper surface: Purple-violet, RHS N82A; Under surface: Violet, RHS 85A-B. Color when opened: Upper surface: Violet, RHS N87B; Under surface: Violet, RHS 85A-B. Disc Florets: None observed.
   Involucres.—Phyllaries: Quantity per inflorescence: About 40 to 50. Length: About 5 mm. Width: About 1 mm. Shape: Lanceolate Apex: Acute. Base: Truncate, fused. Texture, upper and lower surfaces: Setulose edges, verrucose abaxial. Color: Upper and under surfaces: RHS 137A-D.
Reproductive organs:
   Androecium.—Stamen: [symbol] Quantity: [symbol] Anther: [symbol] Shape: [symbol] Length: [symbol] Color: [symbol] Pollen: [symbol] Amount: [symbol] Color: [symbol] .
   Gynoecium.—Quantity per floret: 1. Pistil: [symbol] Shape: [symbol] Color: [symbol] Stigma: [symbol] Shape: Bifurcate Color: White, RHS 155D. Style: [symbol] Length: [symbol] Color: [symbol] Ovary: [symbol] Color: [symbol] .
Seed/fruit: None observed.
Weather tolerance: None observed.
Disease/pest resistance: Not tested.
Disease/pest susceptibility: Not tested.

What is claimed is:

1. A new and distinct cultivar of Aster plant designated ‘MYSTERY LADY MARLENE’, as illustrated and described herein.
